(19) United States
(12) Patent Application Publication
(10) Pub. No.: US 2026/0083920 A1
(43) Pub. Date: Mar. 26, 2026

(54) Split-Luer Lock Connector

(71) Applicant: Conrad A. Flick, III, Bettendorf, IA (US)

(72) Inventor: Conrad A. Flick, III, Bettendorf, IA (US)

(21) Appl. No.: 19/322,359

(22) Filed: Sep. 8, 2025

Related U.S. Application Data
(60) Provisional application No. 63/698,915, filed on Sep. 25, 2024.

(51) Int. Cl. A61M 5/34; A61M 5/32

(52) U.S. Cl. CPC A61M 5/343; A61M 5/3202; A61M 5/3216; A61M 5/344; A61M 2207/00

(57) ABSTRACT

A male Luer coupler supporting partial locking threads facilitates peripheral device coupling to a host device via the coupler.

### FIELD

The present disclosure relates to a coupling arrangement for medical devices and fittings, more particularly medical syringes and access port fittings, and to medical syringes having such a coupling arrangement.

### BACKGROUND

The use of conventional Luer lock coupling arrangements for patient syringes and medical fittings is known. The coupler is integrally molded onto another device using a rotating insert within the molding tool for the thread creation. This coupler comprises a hollow frustum encompassed by an integral, full, hollow cylinder supporting two complete, opposing, internal Luer threads. The syringe barrel typically comprises a one-piece, rigid molding with a closed, distal wall having a central, fluid passageway through an integral, male Luer coupler. A female, Luer cannular hub assembly supporting a distal cannula is threaded into the male Luer coupling cylinder conjoining their singular, conic surfaces with the hub lugs frictionally engaging the Luer male threads for a fluid-tight, secure attachment. The body of most medical fittings is an assembly of injection molded components due to the complexity of creating the male Luer coupling and internal valving details. Typically, the fitting's main body supports the male Luer coupler and a second molding supports the opposing end's female Luer connector. The components are conjoined with adhesive, entrapping the valve member.

### SUMMARY

In one instance, a medical syringe for safely and easily administering a fluid injectant into a patient or assisting their medical procedure is provided. In another instance, an access port for an IV-line set provides one way flow protection. More particularly, the present disclosure relates to single-use devices and fittings which provide greater user efficiency, safety, and sustainability with improved coupling and decoupling means with hubs of various functional tips, both Luer slip and Luer lock. For syringes, the male Luer coupling means may be a molding with external and/or internal thread details inserted into an extruded tube barrel, or a simple, distal, peripheral extension of the injection molded barrel's rim. For valved fittings, the male Luer coupling means is integrally molded about the fitting body with an elastomeric stem inserted to create check valve and pressure relief features. To couple a syringe or valved fitting to a standard, female Luer hub, one simply slips the hub into the male Luer coupler with its lugs passing within the thread voids or passed the directionally yielding, opposing, partial Luer threads, then frictionally coupling to the fluid passageway hollow frustum. In the case of a Luer lock arrangement, a fraction of a twist may be needed for a locked engagement. In all cases, their appearances will be similar to a common syringe or fitting and easily adaptable to current administering techniques.

One embodiment of a Luer slip/lock coupler for syringes according to the present disclosure provides an extruded, tubular barrel distally closed with a male, Luer coupler insert. The coupler consists of two opposing half cylindric sleeves, each supporting an upper, locking, partial thread and a lower, ejector, partial thread with a decoupling peripheral lever, all encircling a central, hollow frustum. The circumferential spacings and profiles of the partial locking threads create opposing thread voids to provide the Luer hub lugs clearance during coupling and uncoupling improving usage efficiency. The female, Luer coupler is a stepped, conical hub transitioning into a cylindrical socket distally supporting a cannula. Two opposing, locking lugs are located on the periphery of its basal rim and frictionally engage the male Luer locking partial thread for securement. For recycling after usage, the female Luer coupler is dislodged from the male Luer coupler by pressing the peripheral levers against the ridge about the sharps container's opening. The coupler's ability to conveniently separate the canula from the body greatly enhances sustainability.

Another embodiment shows the coupler's scalability for larger volume syringes. The distal, body insert molding supporting the male Luer coupler has larger seal and alignment flanges to accommodate the larger diameter barrel. The tubular plunger's features are correspondingly larger as well.

Another embodiment uses an injection molded syringe body and a different approach to the single-axis, butterfly molding of the insert to provide internal and external features on the opposing, cylindric Luer sleeves. The individual sleeves are molded as separate peripherals about the syringe's distal end attached via biased, living hinges. Upon ejection, the halves are conjoined about the integral frustum passageway to complete the coupler. The coupler's internal features are opposing, thread pairs to engage the lugs of the female Luer hub for seal engagement and hub ejection. The external features are opposing threads for accessory attachment, such as a sealing cap. This coupler design and manufacturing process provide the syringe with use efficiency and product sustainability.

Another embodiment of a male Luer coupler according to the present disclosure is for fitting connectors for IV access ports providing one way flow and pressure protection. The proximal, male, slip/lock, Luer coupler consists of opposing, locking, partial threads permitting female Luer hubs to lock-couple with only a partial rotation, avoiding line entanglement. The distal end supports a female, Luer coupler about a passageway selectively occluded with an elastomeric, stem, check valve.

The difficulties and limitations suggested in the preceding and desired features are not intended to be exhaustive but rather are among many which may tend to reduce the effectiveness and user satisfaction with previously known couplers and related devices. Other noteworthy problems and limitations may also exist; however, those presented above demonstrate that conventional syringes admit to worthwhile improvement.

One preferred embodiment is a male Luer coupler insert distally closing the body of a syringe utilizing extruded tubing. Extruded tubing permits a barrel sidewall to have a uniform thinness and a consistent interior diameter with no molding draft to affect the plunger sealing engagement nor its sliding resistance. The tubular body must have sufficient axial rigidity to assist cannular penetration of the patient, and resiliency to maintain the fluid seal with the plunger's distal, rigid, seal ring. The open, proximal, circular end of the body is thermally flanged with a noncircular perimeter to maintain the body's interior configuration, prevent rolling when placed on a surface, and provide gripping means for a user. Both the proximal and distal ends of the syringe barrel are swaged to assist the introduction of the plunger and the molded insert into the barrel, respectively. The body tube is distally closed with a molded insert having a sealing disc with a frustum shaped, fluid passageway through a novel, male Luer coupler arrangement. The locking portion of this coupler consists of two opposing, semi cylindric sleeves, each supporting an upper, locking, partial thread flight and an axially offset, lower, ejector, partial thread flight, and a peripherally extending lever arm/flange to impart an outward motion to the cylindric sleeves for selective, female, Luer hub decoupling. The upper, partial thread flights are circumferentially offset to create two, opposing, thread voids. Both pairs are circumferentially offset to permit single axis molding. When inserting a female Luer hub, the lugs of the female Luer cup may pass within the thread voids within the cylindric walls or be press passed the inwardly sloping thread flanks within the yielding, Luer cylindric sidewalls, and with a slight twist fully engage the locking threads. Frictional coupling of their mating conical tapers of the hollow frustum and female Luer hub results in a fluid seal. For disposal, the two outward opposing lever members engage the perimeter of the sharps container's top surface opening to compromise the columnar integrity of the cylindric Luer sleeves resulting in their outward folding permitting the locking threads to release the lugs and the ejection threads to lift the lugs releasing the female Luer hub from the barrel's frustum. This distortion (kinking) of the sleeves renders the syringe un-reusable. In some applications, the lower, ejector thread may be omitted. An alignment peripheral ring, located slightly distal to the sealing disc, establishes a flexure point for the Luer thread sleeves and is of a slightly larger diameter to close the barrel's proximal swage. The narrow conical taper at the distal frustum end allows a slightly undersized Luer hub the ability to fluidly seal about the frustum with its opposing lugs engaging the leading edges of the opposing securing threads. The outward taper at the base of the frustum grants an oversized Luer hub the same fluid seal about the frustum within the range of the lower limit of the securing thread with the insert's sealing base. The base of the conical opening of a female Luer hub provides a sealing edge for it is free of a parting line or flash due to its molding.

This insert is butterfly (open) molded to allow opposing, partial threads on the inner surfaces of the male, Luer cylindric, half sleeves in a single axis, avoiding timely and costly molding techniques. Upon ejection from the B-side of the mold, initial ejector pins eject and rotate the male Luer cylindric shell alignment ring passed the rotational center of a biased living hinge, snapping the ring closed about the frustum, coming to rest on the sealing disc. The platen's travel is reversed momentarily to compressively seat the ring securely onto the base sealing disc within the biased clamp; then the completed molding is ejected overcoming the seal crown's tooling undercut. Once inserted, the tubular body of the barrel further establishes the cylindrical conjoined nature of the male Luer housing, while allowing slight, lateral flexibility for the halves to receive the female hub lugs over the threads' crests within the first half of the locking thread flight. The Luer/coupling engagement is timed so the female Luer hub seats onto the hollow frustum when lugs engage midway on the partial thread flights, one eighth rotation from the thread start. The contact point of the female Luer hub's lug with the partial thread is directly above the biased living hinge's extended, connecting tendon resisting any thread uplift. The opposing, thread voids also permit the quick separation of the functional Luer tip from the body during a procedure, such as an endovascular guide wire introduction. The frustum's length allows aspirate viewing between its tip and the needle base within the hub. This insert is pressed into the body and secured frictionally by dimensional interference. For additional component retention for elevated internal barrel pressures experienced with high viscosity injectants, other retention methods may include an applied adhesive, or a suitable form of welding, such as with a thermal probe, ultra-sonic vibration, or spin may be used, or a stanchion on the cylindric Luer shell with a complementary aperture in body sidewall.

The plunger has a proximal, thermoformed, integral, finger pad ring and a “tipped”, distal end with an integral, fluid sealing ring. “Tipping” is a process to thermally occlude tubular ends and/or thermally impart a feature on a plastic tube. The fluid seal is established by the resiliency of the barrel sidewall and the insert's rigid, sealing ring. The seal interface is free of any forming imperfections by having the forming receiver in the “B” mold portion having a slight undercut placing the parting line above the seal crown. The integral, lateral, radiating fins of the plunger's extruded, tubular shaft minimize frictional, tubular contact with barrel and ensure its axial travel within the tubular body to maintain the seal ring's fluid tight seal with the resilient, barrel sidewall.

The cannular sheath is pivotally attached to the female Luer hub via a biased living hinge (a flexible hinge). When in the shielding position, its tubular trough is split coupled (yoke) in tension about the cannular socket housing of the Luer hub. This tension is sufficient to provide protection from any lateral deflection exposing the cannular tip and yielding enough to allow re-shielding with a slight swipe with a neighboring surface, ex. counter. Plasticity in the living hinge resulting from being packaged in the closed position will instill a return memory to the sheath hinge. Two finger tabs at the tension yoke assists the opening rotation with finger tips clear of the cannular tip. These tabs have latches integrated on the extreme edge positioned to mate with the recess on the hub lateral extension to lock open the sheath. These tabs may also assist in decoupling the female Luer lock if the threads fail to disengage. One merely rotates the inserted body ⅛^{th} turn while the tabs lock onto the leaves in the disposal opening of the sharp's container to unscrew reducing frictional tension and ejecting. Two continuous, contact lines on the assembly's top surfaces are provided for attachment and sealing with the packaging cover. The packaging enclosure for the female, Luer hub assembly is removed leaving the cover to maintain the sterile field until usage.

Another embodiment shows the scalability of this approach with a similar device having a larger volume barrel. The closed, distal, body insert molding has larger diameter seal and containment flanges for chamber closure and posturing the fluid passageway supporting the same, male, Luer coupler, respectively. The flanges are coupled together with integral, complimentary locking stanchions. The female, Luer cannular assembly is the same. The tubular plunger is simply an enlarged previous version.

Another embodiment features an injection molded body (conventional) and a different molding approach about the distal, peripheral edge with similar functionality. The butterfly molded insert has two, opposingly positioned, semi-cylindric sleeves biasedly hinged about the central frustum passageway, integral with the end wall. This approach enables the partial, internal, thread pairs, locking and ejecting, to be axially aligned and provide the opposing thread voids between the halves. Both halves are pivoted about the biased hinges encircling the hollow frustum by lead ejector pins, then snap locked into complimentary latches about the hollow frustum by the momentarily descending mold platen prior to the body being fully ejected. The contact point of the female Luer hub's lug with the partial locking thread is directly below the biased living hinge's connecting tendon preventing any thread lift during attachment. This is an efficient means to obtain this level of improved functionality while eliminating costly injection mold details, e.g., rotational molding details and core pulls. There are outwardly extending levers/flanges about the insert's distal end which when flexed, force the cylindric sleeves outward releasing the female Luer hub lugs from the locking thread and levering free the hub with the lower, ejection thread. This component separation enhances complete recycling. This molding approach further facilitates molding partial, external threads on the cylindric sleeves for the attachment of various accessories, such as caps. This process permits apertures and/or stanchions for securing components or providing flushing access to recesses created by the Luer shell and frustum passageway to be incorporated. The plunger has an injection molded cruciform shaft with a proximal, integral, noncircular, finger pad and a frictionally attached elastomeric grommet attached to its distal end providing a fluid seal with a distal, circumferential bead.

These embodiments resemble typical, Luer lock syringes in appearance with improved functionality. The material selection will be based on biocompatibility, functionality, and sustainability.

The components and their elements of the various embodiments may be interchanged. The cross-section of the barrel and the related, plunger elements may be circular, elliptical, or irregular depending on usage and sealing means.

On the body, there are graduated indicia to determine the volume within the fluid compartment in relation to the plunger's seal position within the syringe's body.

Another embodiment of the disclosure which is intended to address concerns and accomplish at least some of the foregoing objects comprises a male Luer coupler disclosure for fitting connectors for IV access ports providing check valve and pressure protection. This male, Luer coupler on the fitting's distal end consists of two opposing, rodded, partial sleeves supporting partial, locking threads encircling a central, hollow frustum. These partial sleeves are pivotally positioned with bias about the body on radiating stanchions to accept the female Luer lugs pressed into the inwardly sloping partial thread flanks and locking with a fractional rotation. Also, the circumferential spacing of the partial threads creates opposing, axial thread voids allowing female Luer coupler lugs clear passage when coupling and decoupling without line entanglement associated with current Luer couplings. The opposing proximal end supports a female, Luer coupler about a passageway selectively occluded with an elastomeric, stem check valve. The passageway's cylindric throat region compresses the elliptical valve's fluid channel in the rest position creating seals internally and circumferentially. The insertion of another male Luer member axially and distally compresses the valve member into the elliptical chamber distorting the valve channel to open the orifice permitting fluid flow through the port fitting. The interior geometry of the housing proximally transitions from elliptical to cylindrical with the same cross-sectional area creating a shoulder stop and permitting molding core retraction. Retention of the elastomeric valve is based on its material's durometer to resist reshaping from elliptical to cylindrical. This deformation occurs at the axial pressure greater than an elevated systolic blood pressure and lower than one causing venular rupture.

The difficulties and limitations suggested in the preceding and desired features are not intended to be exhaustive but rather are among many which may tend to reduce the effectiveness and user satisfaction with previous known syringes and medical fittings. Other noteworthy problems and limitations may also exist; however, those presented above should be sufficient to demonstrate that syringes appearing in the past will admit to worthwhile improvement.

### BRIEF DESCRIPTION OF THE DRAWINGS

Other objects and advantages of the present disclosure will become apparent from the following detailed description of the preferred embodiments thereof taken in conjunction with the accompanying drawings wherein:

FIG. 1 is an axonometric view of a syringe utilizing a Luer coupling in accordance with one embodiment of the disclosure;

FIG. 2 is an exploded, axonometric view of the syringe of FIG. 1 with a retracted sheath;

FIG. 3 is a longitudinal, cross-sectional, side view of the syringe of FIG. 1;

FIG. 4 is a detailed view showing a Luer lock coupling arrangement for the syringe of FIG. 1;

FIG. 5 is an axonometric, side view of a male Luer coupler insert;

FIG. 6 is a longitudinal, top view of the of the insert of FIG. 5;

FIG. 7 is an internal, side view of the insert of FIG. 5;

FIG. 8 is a longitudinal, side view of a male Luer coupler insert in its open molded state revealing single axis molding;

FIG. 9 is an axonometric, top view of the male Luer coupler insert in FIG. 8;

FIG. 10 is a top view of the male Luer coupler insert in FIG. 8;

FIG. 11 is a bottom view of the male Luer coupler insert in FIG. 8;

FIG. 12 is an axonometric view of an extruded tubular plunger in FIG. 1;

FIG. 13 is a detailed view the “tipped” seal region of the plunger in FIG. 1;

FIG. 14 is a longitudinal, top view of the needle hub and sheath in FIG. 1;

FIG. 15 is an axonometric view of a needle hub in FIG. 14 with a retracted sheath;

FIG. 16 is a front view of the needle hub and sheath in FIG. 14;

FIG. 17 is an axonometric view of a needle hub in FIG. 14 with a detached sheath.

FIG. 18 is an axonometric view of a larger, male Luer coupler insert for a larger capacity syringe;

FIG. 19 is a side, cross-sectional, side view of the insert of Claim 18;

FIG. 20 is an additional, side, cross-sectional, side view of the insert of Claim 18;

FIG. 21 is an axonometric, top view of a larger male Luer coupler insert in its open molded state;

FIG. 22 is an axonometric, side view of a larger male Luer coupler insert in its open molded state;

FIG. 23 is an axonometric view of an injection molded syringe with in integral, male Luer coupler in accordance with this disclosure;

FIG. 24 is an axonometric, top view of the integral male Luer coupler of FIG. 21;

FIG. 25 is a detail, side, cross-sectional view of the coupler of FIG. 21.

FIG. 26 is a top view of the syringe with a male Luer coupler insert in its open molded state revealing single axis molding;

FIG. 27 is a detailed view of the syringe with a male Luer coupler insert in its open molded state revealing single axis molding;

FIG. 28 is a top view of the syringe body in FIG. 23 in its molded state revealing single axis molding;

FIG. 29 is a bottom view of the male Luer coupler in FIG. 23.

FIG. 30 is an axonometric view of the fitting supporting a novel, male Luer coupler arrangement in accordance with the disclosure;

FIG. 31 is an axonometric view of the fitting supporting a novel, male Luer coupler;

FIG. 32 is an exploded, axonometric, longitudinal view a fitting application utilizing a male Luer coupler;

FIG. 33 is a top view of the fitting of FIG. 30 revealing the securing hardware for both the male and female Luer couplers;

FIG. 34 is a bottom view of the fitting of FIG. 30 revealing the elastomeric member positioned within a female Luer coupler;

FIG. 35 is a side, cross-sectional, side view of the fitting of Claim 28 along the A-axis;

FIG. 36 is a side, cross-sectional, side view of the fitting of Claim 28 along the B-axis;

FIG. 37 is an axonometric view of the fitting's valve chamber;

FIG. 38 is an axonometric view of the elastomeric member at rest;

FIG. 39 is an axonometric, cross-sectional view of the elastomeric member of FIG. 38;

FIG. 40 is an axonometric view of the elastomeric member within the fitting of FIG. in its compressed state;

FIG. 41 is an axonometric, cross-sectional view of the fitting of FIG. 30 with the elastomeric member in its axially compressed state;

FIG. 42 is an alternative embodiment of the elastomeric member within the body in FIG. 36;

FIG. 43 reveals the elastomeric member of FIG. 42 in its configured state within the fitting;

FIG. 44 shows a longitudinal cross-section of the elastomeric member in FIG. 43;

FIG. 45 reveals the male Luer coupler integrally molded with a syringe body;

FIG. 46 is a cross-sectional view of the syringe in FIG. 45.

FIG. 47 is a detailed, section view of the distal end of the syringe in FIG. 45 with a positioned peripheral Luer hub.

FIG. 48 is a top view of the syringe in FIG. 45.

FIG. 49 is a bottom view of the syringe in FIG. 45.

FIG. 50 is an axonometric view of the extruded plunger from the syringe in FIG. 45.

FIG. 51 is a cross-sectional view of the plunger in FIG. 50.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and made part of this disclosure.

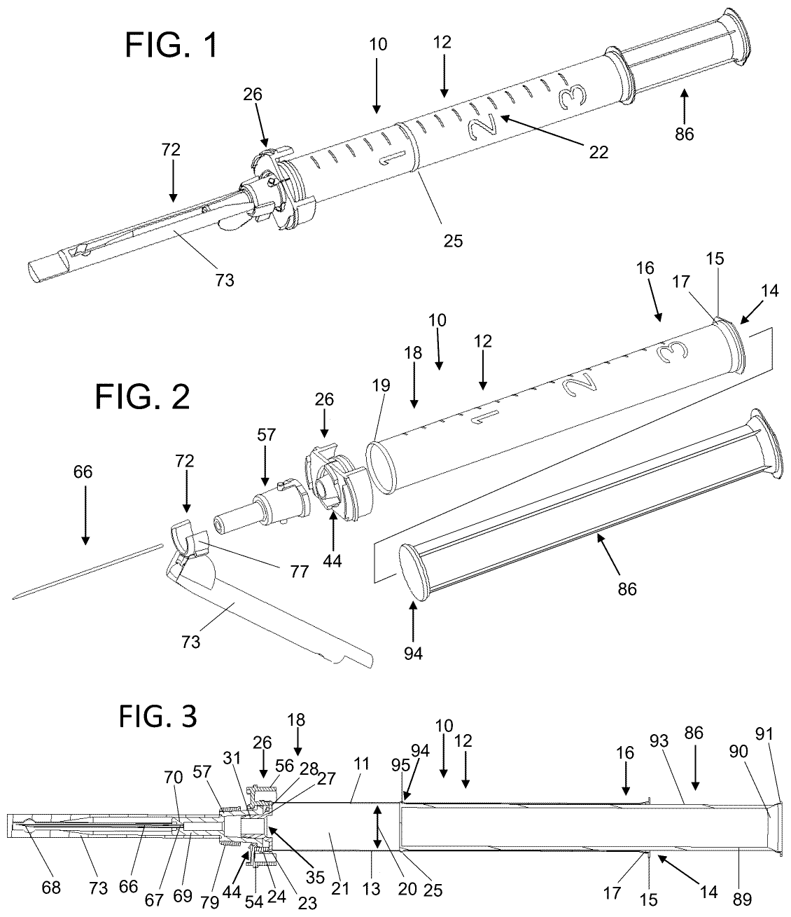

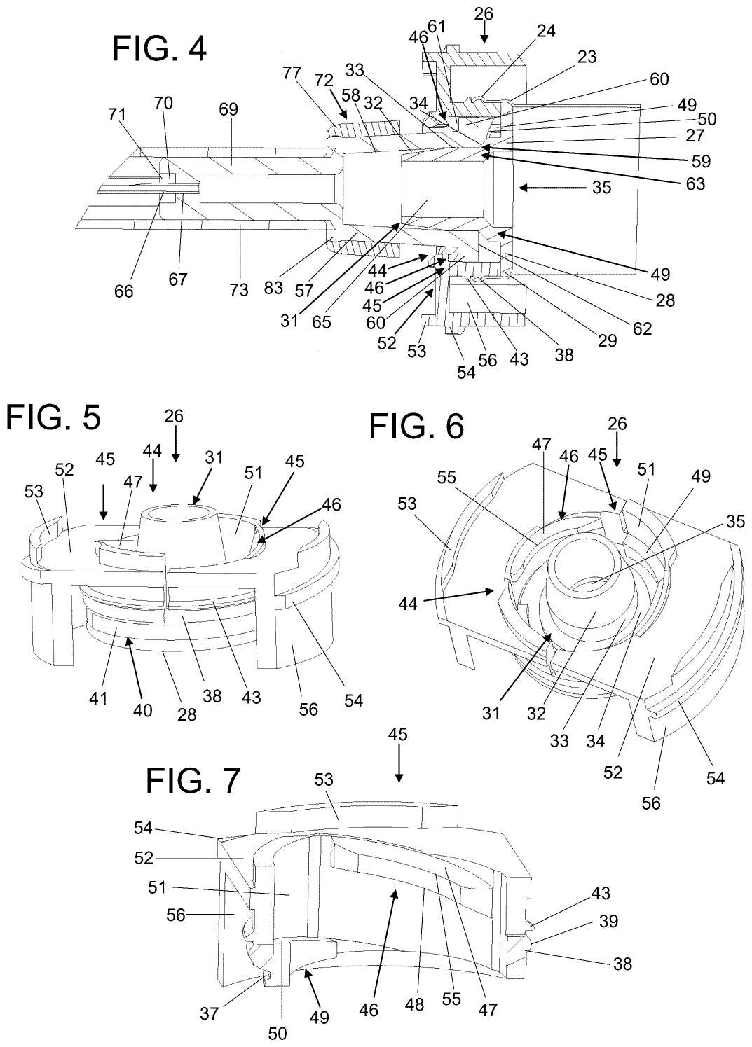

Referring now particularly to the drawings revealing three approaches for the same concept, wherein like reference characters refer to like parts, and initially to FIGS. 1, 2, and 3, there will be seen axonometric and cross-sectional views of a syringe 10 utilizing insert 26 which supports novel male Luer coupler 44 in accordance with one preferred embodiment of the disclosure. The syringe 10 comprises a tubular body 12 having a resilient sidewall 13 with a uniform thinness and a consistent interior diameter 20. The tubular body 12 must have sufficient axial rigidity to assist cannular 66 penetration of the patient, and resiliency to maintain the fluid seal with the plunger 86's distal, rigid, seal flange 94. The open, proximal, circular end 14 of the body 12 is thermally flanged 15 with a noncircular perimeter to maintain the body's 12 tubular configuration, prevent rolling when placed on a surface, and provide gripping means for a user. Both the proximal and distal ends, 16 and 18 respectively, of the syringe body 12 are swaged 17 and 19, respectively, to assist the introduction of the plunger 86 and the molded insert 26 into the barrel 12. FIG. 4 reveals the body tube 11 is distally closed with a molded insert 26 having a sealing base 27 supporting base seal disc 28 with a hollow frustum 31's fluid passageway 35 through a novel, male Luer coupler arrangement 44. FIGS. 5, 6, and 7 show the locking portion of this coupler 44 within insert 26 consists of two opposing, semi cylindric sleeves 45, each supporting an upper, locking, partial thread flight 46 and an axially and circumferentially offset, lower, ejector, partial thread flight 49, and a peripherally extending lever arm/flange 52 to impart an outward motion to the cylindric sleeves 45 for female Luer assembly 57 disengagement. The upper, partial, locking thread flights 46 are circumferentially offset to create two, opposing, thread voids 51 for receiving Luer hub lugs 60 upon insertion. The locking thread 46 is a simple buttress style thread providing a sloping upper flank 47 for lug 60's selective passage upon insertion and lower square flank 48 for frictional threaded engagement with topside 61 of lug 60. The ejector thread 49 has an upper square thread flank 50 engages the bottom side 62 of lug 60 for selective ejection when disposing. When inserting the female Luer assembly 57, the lugs 60 may pass within the thread voids 51 within the cylindric sleeves 45 or be press passed the inwardly sloping thread flanks 47 within the yielding, Luer cylindric sleeves/sidewalls 45, and with a slight twist fully engage the locking threads 46. Frictional coupling of their typical, mating conical tapers 33 and 58 of the hollow frustum 31 and female, Luer hub 57, respectively, results in a fluid seal. For disposal, the two catch stanchions 53 of the outward opposing lever members 52 engage the perimeter of the sharps container's top surface opening to compromise the columnar integrity of the cylindric Luer sleeves 45 resulting in their outward folding permitting the locking threads 46 to release the lugs 60 and the ejection threads 49 to rotate and lift the female Luer hub 57 via the bottom sides 62 of lugs 60 from the insert 26's hollow frustum 31. This distortion of the sleeves 45 renders the syringe 10 un-reusable. In some applications, the lower, ejector thread 49 may be omitted. The hollow frustum 31's varying tapers accommodates dimensional variances in the tapers of peripheral devices. The narrow conical taper 32 at the distal end of frustum 31 allows a slightly undersized Luer hub the ability to fluidly seal about the frustum 31 with its opposing lugs engaging the leading edges of the opposing locking threads 46. The typical taper 33 of the frustum 31's midsection frictionally couples with the hub's typical internal taper 58. The outward taper 34 at the base of the frustum 31 grants a fluid seal with an oversized Luer hub's rim about the frustum 31 within the range of the lower limit of the insert base 28. The base rim 59 of the conical opening 63 of a Luer hub 57 provides a sealing edge for it is free of a mold parting line or flash due to its molding.

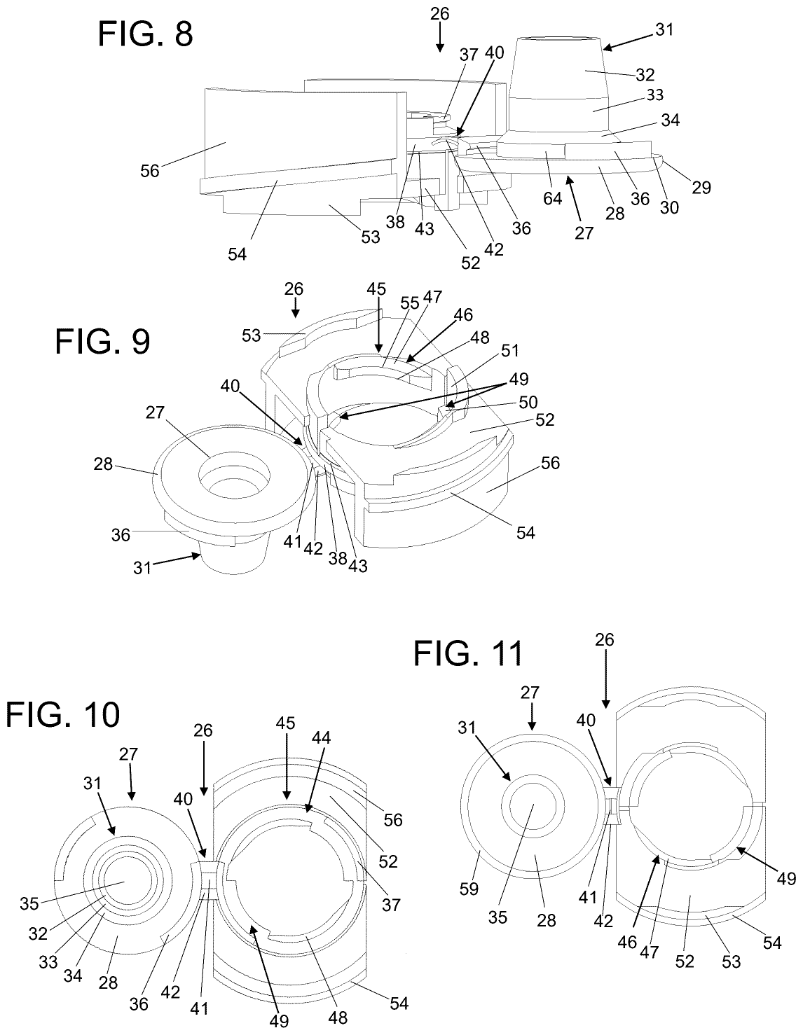

Referring now to FIGS. 8, 9, 10, and 11, the insert 26 is butterfly (open) molded to allow opposing, partial, locking threads 46 on the inner surfaces of the male, Luer cylindric, half sleeves 45 to be created segmented in a single axis, avoiding timely and costly molding techniques. Upon ejection from the B-side of the mold, initial engaging ejector pins eject and rotate the male Luer coupler 44 passed the rotational center of a biased living hinge 40, its biased tendons 42 snapping the coupler 44 closed about the frustum 31, coming to rest on the sealing disc 28. The platen's travel is reversed momentarily to compressively seat the coupler assembly 44 securely onto the base sealing disc 28 with its biased, internal, male clasp 36 fully engaging the biased external, female clasp 37 of the insert base 27; then the completed molding 26 is popped out (ejected) overcoming the seal interface crown 29's tooling undercut. The seal interface crown 29 is free of any forming imperfections by having the forming receiver in the “B” mold portion having a slight undercut placing the parting line 30 above it. Once inserted, the barrel tubular body 12 further establishes the conjoined, cylindrical nature of insert 26, while allowing slight, lateral flexibility for the sleeves 45 to receive the female hub lugs 60 over the crests 55 of inwardly sloping thread flanks 47 within the first half of the locking thread flight 46. The Luer coupling 44's engagement is timed so the female Luer hub 57 seats onto the frustum 31 when lugs 60 engage midway on the partial, locking thread flights 46, one eighth of a full rotation from the thread start. The contact surface 61 of the female Luer hub 57's lug 60 with the lower square thread flank 48 of partial, locking thread 46 is directly above the biased living hinge 40's extended, connecting tendon 41 resisting any thread uplift. The opposing voids 51 also permit the quick separation of the functional Luer tip 56 from the barrel 12 during a procedure, such as an endovascular guide wire introduction. The hollow frustum 31's length 65 allows aspirate viewing between its base 64 and the canular (needle) base 67 within the hub 57's cannular socket 70. The narrow, fluid passageway 35 minimizes the negative space associated with medicant delivery. The alignment flange 38 is a semi-circular band wrapping the sleeve's 45 base to co-establish the insert 26's axial alignment. The crown 39 of this band 38 protrudes peripherally beyond the insert base 28 seal crown 29 to close the body 12's distal swage 19. This insert 26 is pressed into the body 12 and secured frictionally by dimensional interference. Sidewall 13 abuts a stop 43 projecting radially on its distal side of the crown 39. For additional component retention for elevated internal barrel pressures experienced with high viscosity injectants, other retention methods may include an applied adhesive, or a suitable form of welding, such as thermal, ultra sonic vibration, or spin may be used, or a stanchion on the cylindric insert 26 with a complementary aperture in body 12's sidewall 13.

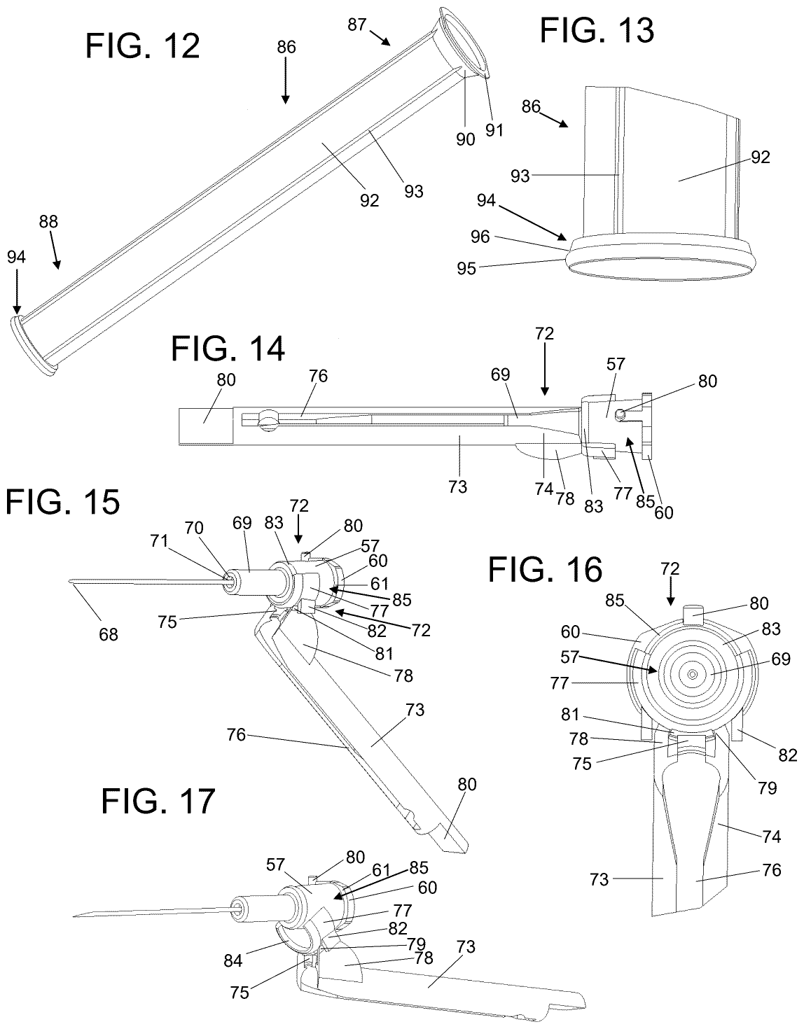

Referring to FIGS. 12 and 13, the plunger 86 comprises a tubular shaft 92 with a proximal 87, thermoformed, integral, finger pad ring 91 and a “tipped”, distal end 88 with an integral, fluid sealing flange 94. “Tipping” is a process to thermally occlude tubular ends and/or thermally impart on a plastic tube 11 a feature, such as the sealing flange 94. The fluid seal is established by dimensional interference of the rigid, sealing flange 94's interface crown 95 and the resilient barrel sidewall 13. The seal interface crown 95 is substantially free of any forming imperfections by having the forming receiver in the “B” mold portion having a slight undercut placing the parting line 96 above and within the seal crown 95's diameter. The integral, lateral, radiating fins 93 of the plunger's extruded, tubular shaft 92 minimize frictional, tubular contact with the barrel 11 and ensure its axial travel within the tubular body 12 to maintain the fluid tight seal of the seal flange 94's crown 95 with the resilient, barrel sidewall 13. The shaft 92 is proximally swaged 90 below finger pad 91 to establish a chamber seal with body 12's proximal swage 17 when fully engaged.

Now referencing FIGS. 14, 15, 16, and 17, the cannular sheath assembly 72 is rotationally and tensionally attached to the external, tapered side 85 of the female Luer hub 57 via a tensioned hub yoke 77 retained by the hub's retention bead 83 engaging the yoke's circular groove 84. The canular trough 76 is selectively, rotatably attached by curved, biased living hinge 75 to hub yoke 77. When in the shielding position, the canular trough 76 is secured by the sheath 73's tensioned yoke 74 about the cannular socket housing 69 of the Luer hub 57. This tension is sufficient to provide protection from any lateral deflection exposing the cannula 66 and its sharp tip 68, yet yielding enough to allow re-shielding with a slight swipe with a finger or a neighboring surface, ex. counter. The curved profile of living hinge 75, along with plasticity resulting from being packaged in the closed position, will instill a return memory to the sheath 73. Sheath rotation is initiated by downward finger pressure applied to the distal finger pad 80 of sheath 73. Two finger tabs 78 at the tension sheath yoke 74 assists the opening rotation with finger tips clear of the cannular tip 68. These tabs 78 have latches 79 integrated on the extreme edge positioned to mate with the latch receiver 81 on the hub 57 to hold open the sheath 73. These tabs 78 may also assist in decoupling the hub 57 if the locking threads 46 fail to disengage. One merely rotates the inserted body 12 ⅛^{th} turn while the tabs 78 lock onto the leaves in the disposal opening of the sharp's container to unscrew. The sheath 73 may be rotated about the hub 57's retention bead 83 to prevent procedural interference. To remove the sheath assembly 72 entirely, rotate past the open stops 82, expanding the mounting hub yoke 77 to release from the retention bead 83. To reattach, simply snap back into place. Two continuous, contact lines on its top surfaces are provided for attachment and sealing with the cover of the packaging enclosure. The packaging enclosure for the female, Luer hub assembly 57 is removed leaving the cover to maintain the sterile field until usage.

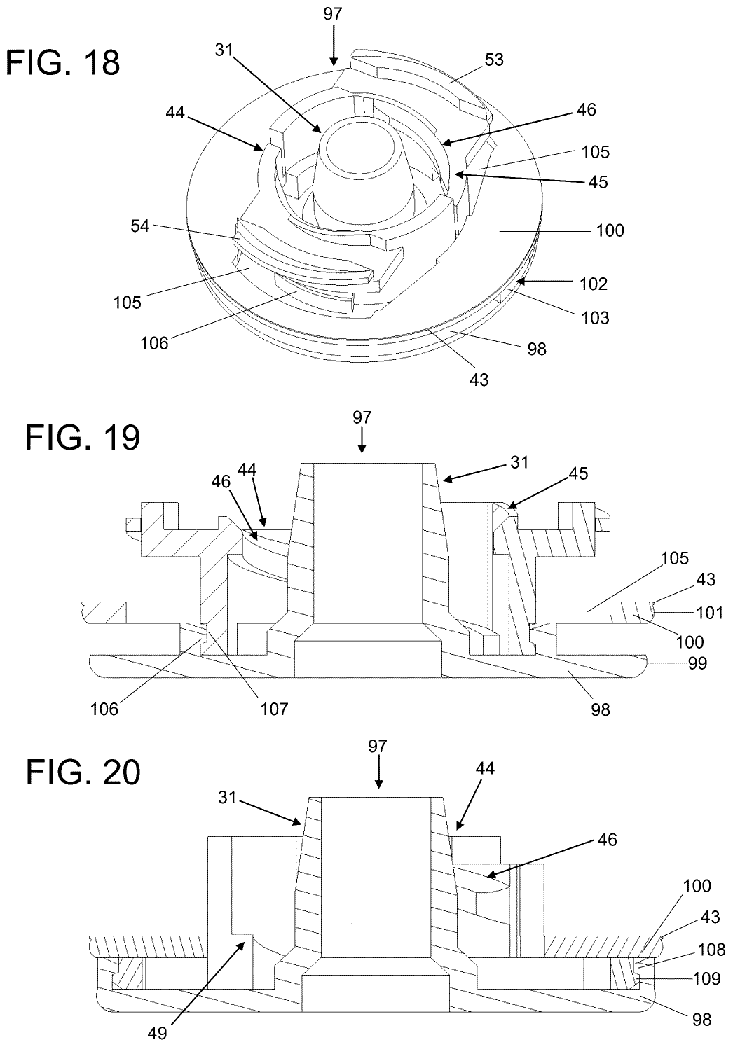

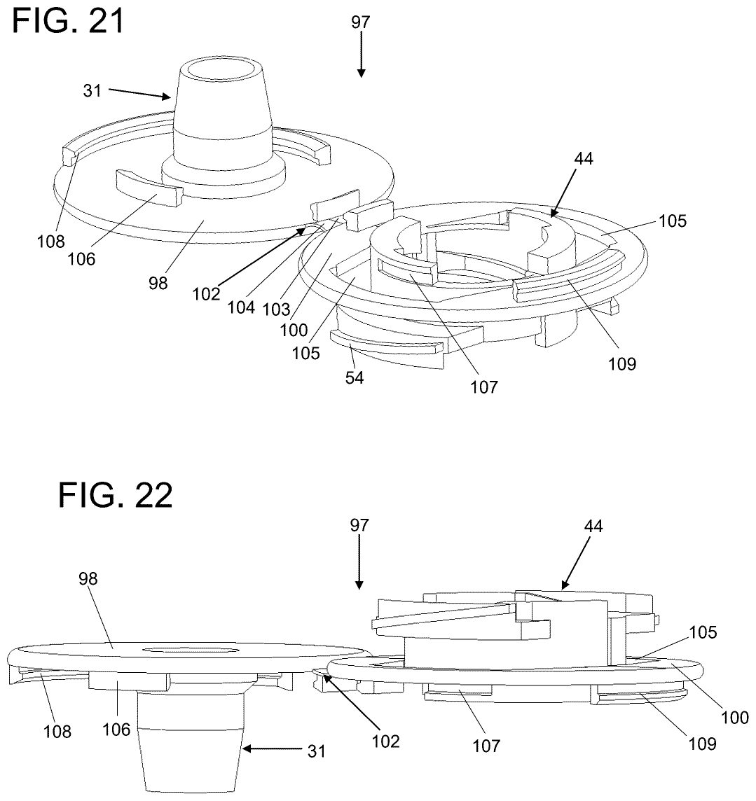

FIGS. 18, 19, and 20 reveal a larger diametric insert 97 supporting a similar male Luer coupler 44 having larger diameter seal and alignment flanges 98 and 100, respectively, for chamber closure and posturing the fluid passageway 35 for larger volume syringes. FIGS. 21 and 22 display the single axis, butterfly molded piece 97 with opposing partial locking threads 46 and ejector threads 49 on inner surfaces of the male cylindric half sleeves 45. Upon ejection from the mold, the halves are partially conjoined about the biased, living hinge 102's hinge tendon 103 by the biased tendon 104; and then with a momentarily reversing platen, the integral, complimentary, internal and external, locking stanchions 106 and 107, 108 and 109, respectively, lockingly engage. The inner locks engagement occurs in direct alignment with the engagement of the locking thread 46 with the Luer hub lug 60 preventing any uplifting by the later engagement. This later engagement ensures the lock with the hub lug 60 resisting inward movement of the internal female lock 107. Alignment flange 100's openings 105 permit the external thread 54 molding and clearance for the Luer sleeves 45 during hub ejection.

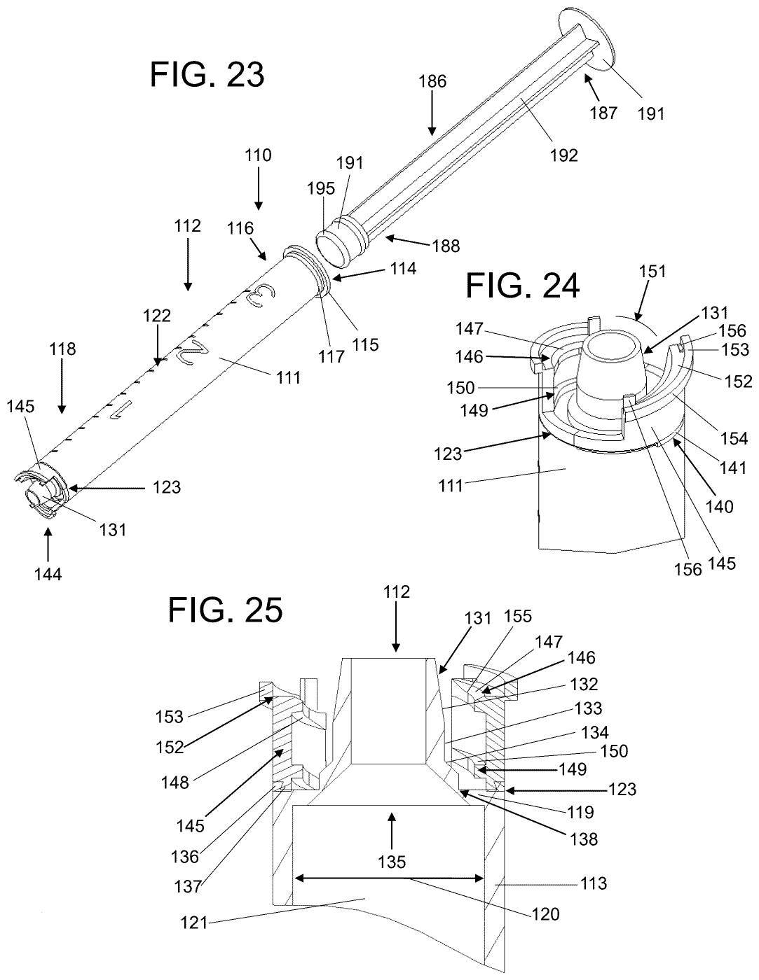

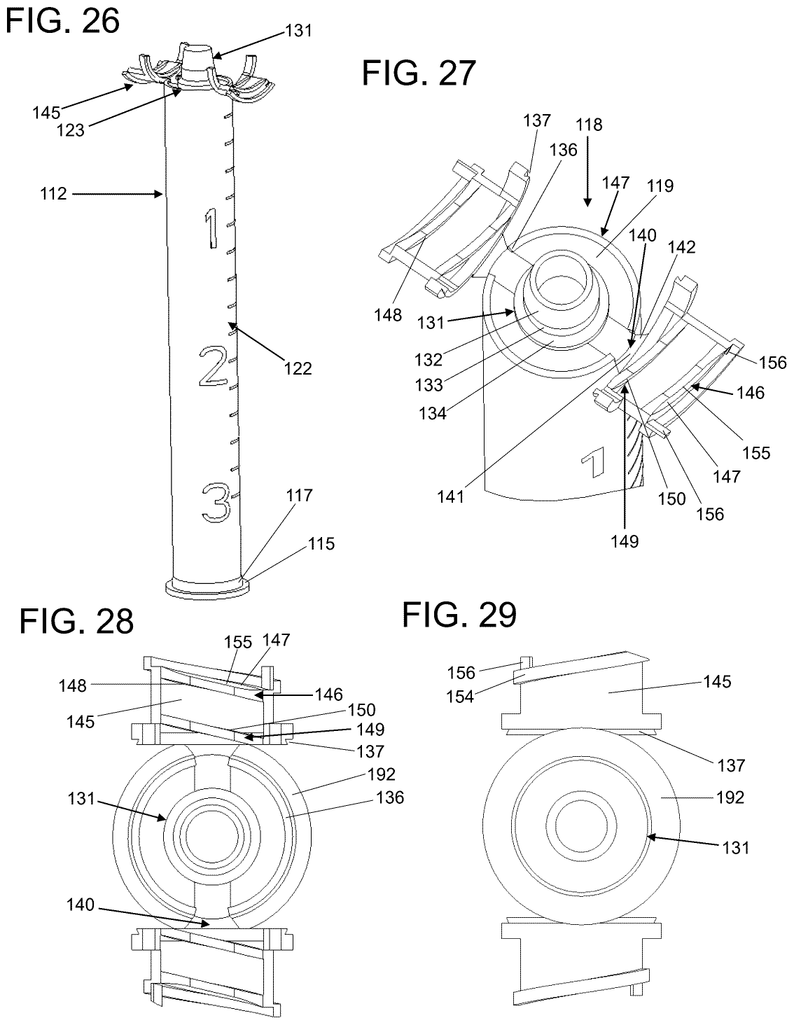

In another embodiment, FIG. 23 is an axonometric view of an injection molded body 111 (conventional) with a male, Luer lock assembly 144 butterfly-molded to periphery of the distal end 118 with an accompanying plunger 186. Butterfly-molding permits segmented, internal, locking and ejecting thread details 146 and 149, respectively, to be molded axially aligned within the Luer cylindric sleeves 145 in a single axis. In FIGS. 24 and 25, there will be seen axonometric and cross-sectional views of a male, Luer lock assembly 144 in accordance with one preferred embodiment of the disclosure. The syringe 110 comprises an injection molded body 112, having a sidewall 113 with a varied thickness from core drift during injection molding and a distally diminishing interior diameter 120 due to molding core draft. Typically, the plunger 186 utilizes an elastomeric grommet 194 to overcome these diametric variations 25 and establish its seal within the fluid chamber 121. The open, proximal 116, circular end 114 of the body 112 supports an integral flange 115 with a noncircular perimeter to maintain the body's 112 configuration, prevent rolling when placed on a surface, and provide gripping means for a user. The proximal end 116 is outwardly stepped 117 to assist the plunger 186 insertion. The closed distal end 118 supports a hollow frustum 131 shaped, fluid passageway 135 integrally molded into the distal end wall 119 and passing through a novel, male Luer coupler arrangement 144. The locking portion of this coupler 144 consists of two opposing, semi cylindric sleeves 145, each supporting an upper, locking, partial thread flight 146 and an axially aligned, lower, ejector, partial thread flight 149, and from its distal rim 123 a peripherally extending lever arm/flange 152 to impart an outward motion to the cylindric sleeve 145 to initiate female, Luer assembly decoupling. The upper, locking, partial thread flights 146s' geometry and circumferential offsets create two, opposing, openings 151. Referring to FIGS. 26,27,28, and 29, the butterfly (open) molding orientation of the Luer sleeves 145 permit the pairs of partial, thread flights 146 and 149 to be in axial alignment with single axis molding. This molding approach also facilitates molding partial, external threads 154 on the cylindric sleeves 145 for the attachment of various accessories, such as caps. This provides many economies in hardware sophistication, molding techniques, including dwell time, and material. When inserting a female Luer assembly, its lugs may pass within the openings 151 or be press passed the thread crests 155 of the inwardly sloping thread flanks 147 within the yielding, Luer cylindric sleeves/sidewalls 145, and with a slight twist fully engage the locking threads 146. The opposing openings 151 also permit the quick decoupling of the functional Luer tip from the barrel 112 during a procedure, such as an endovascular guide wire introduction. Frictional coupling of the conical tapers 133 of the hollow frustum 131 and a female, Luer hub's interior taper or proximal, base rim results in a fluid seal. The hollow frustum 131's varying tapers accommodates dimensional variances in the tapers of peripheral devices. The narrow conical taper 132 at the distal end of frustum 131 allows a slightly undersized Luer hub the ability to fluidly seal about the frustum 131 with its opposing lugs engaging the leading edges of the opposing locking threads 146. The typical taper 133 of the frustum 131's midsection frictionally couples with the hub's typical internal taper 158. The outward taper 134 at the base of the frustum 131 grants a fluid seal with an oversized Luer hub's rim about the frustum 131 within the range of the lower limit of the end wall 119. The base rim of the conical opening of a Luer hub provides a sealing edge for it is free of a mold parting line or flash due to its molding. For disposal, the two catch stanchions 153 of the outward opposing lever members 152 engage the perimeter of a sharps container's top surface opening to compromise the columnar integrity of the cylindric Luer sleeves 145 resulting in their outward folding permitting the locking threads 146 to release the lugs and the ejection threads 149 to lift the female Luer hub via its lugs from the frustum 131. This distortion of the sleeves 145 renders the syringe 110 un-reusable. In some applications, the lower, ejector thread 149 may be omitted. If omitted, the Luer hub may be laterally twisted to snap the frustum 131 at its circumferential, thinned segment 138 free from the distal end wall 119 and outwardly forcing the slightly upward sloping thread flanks 148 to release the Luer lugs. This alternative cannular decoupling means may be deployed on any cannular supporting frustum 131.

Upon ejection from the mold's B half, the individual, opposing Luer sleeves 145 are snap rotated about the passageway 135's frustum 131 by the biased tendons 142 of the living hinge 140 and snap locked into position by the momentary, platen travel reversal contacting stanchions 156 to engage the male and female locking clasps 136 and 137 of the distal end wall 119 and the Luer sleeve 145, respectively. The contact point of an approaching, female Luer hub's lug with the slightly upward sloping, lower partial, locking thread 146 is directly above the biased living hinge 140's connecting tendon 141 preventing any thread lift. This molding approach is an efficient means to obtain this level of improved functionality while eliminating costly injection mold details, ex. rotational molding details and core pulls. The plunger 186 is an injection molded cruciform shaft 192 with an integral, noncircular, finger pad 191 about its proximal end 187, and a frictionally attached elastomeric grommet 194 to its distal end 188 providing a fluid seal with a distal, circumferential bead 195. On the body 112, there are graduated indicia 122 to determine the volume within the fluid chamber 121 in relation to the plunger 186's sealing bead 195's position within the syringe's body 112.

These embodiments resemble typical, Luer lock syringes in appearance with improved functionality and sustainability. The material selection will be based on biocompatibility, functionality, and sustainability.

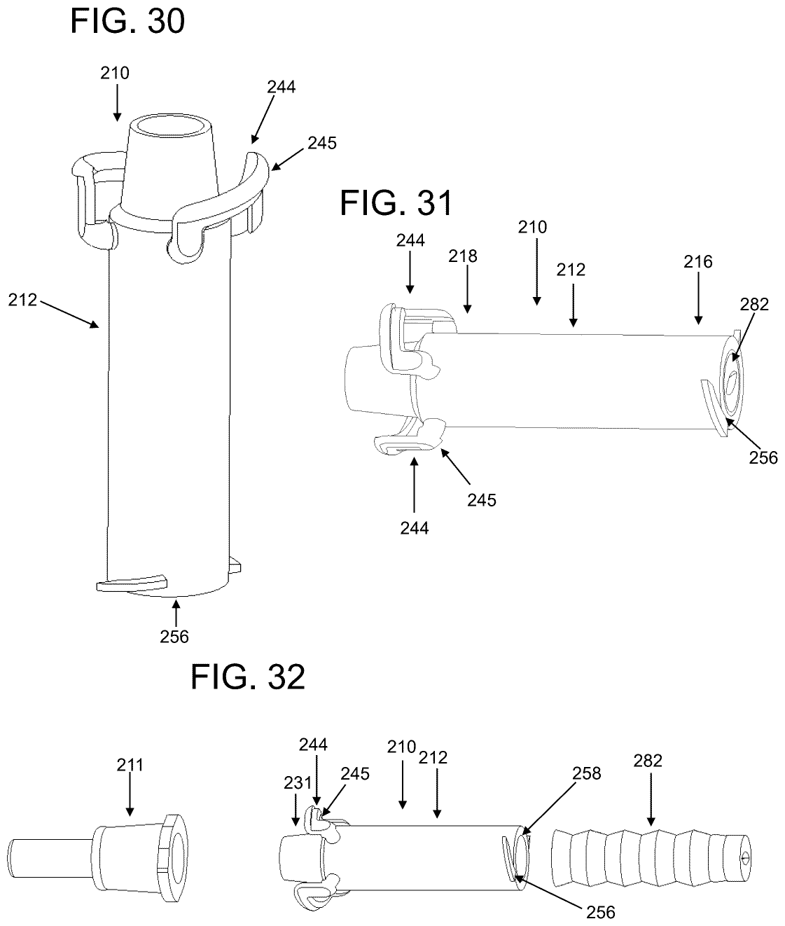

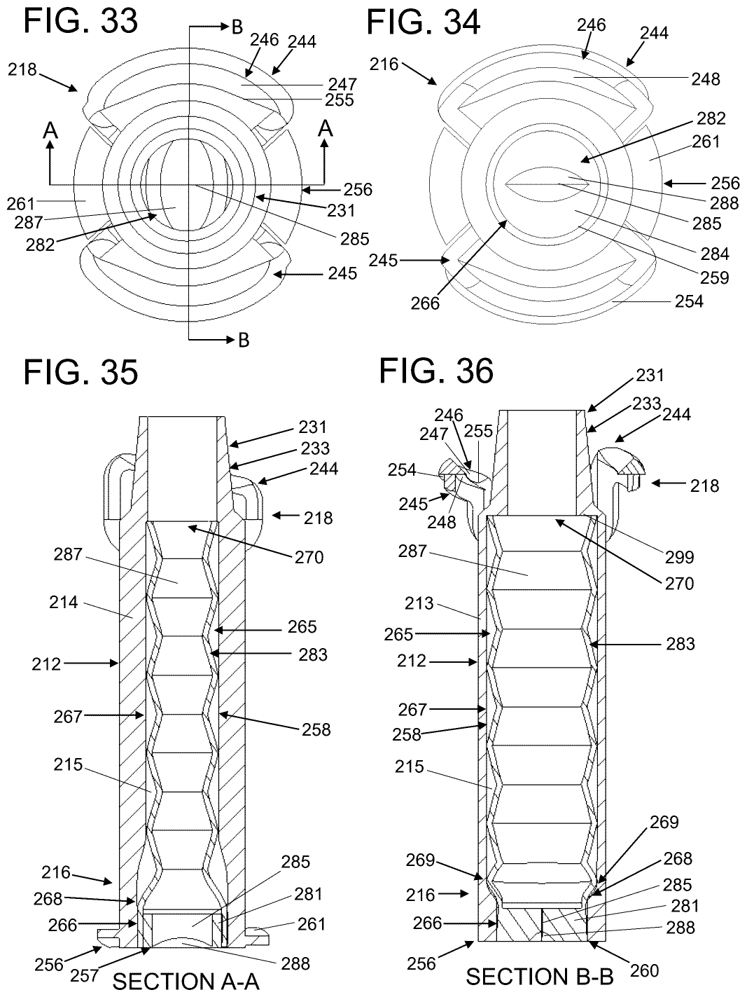

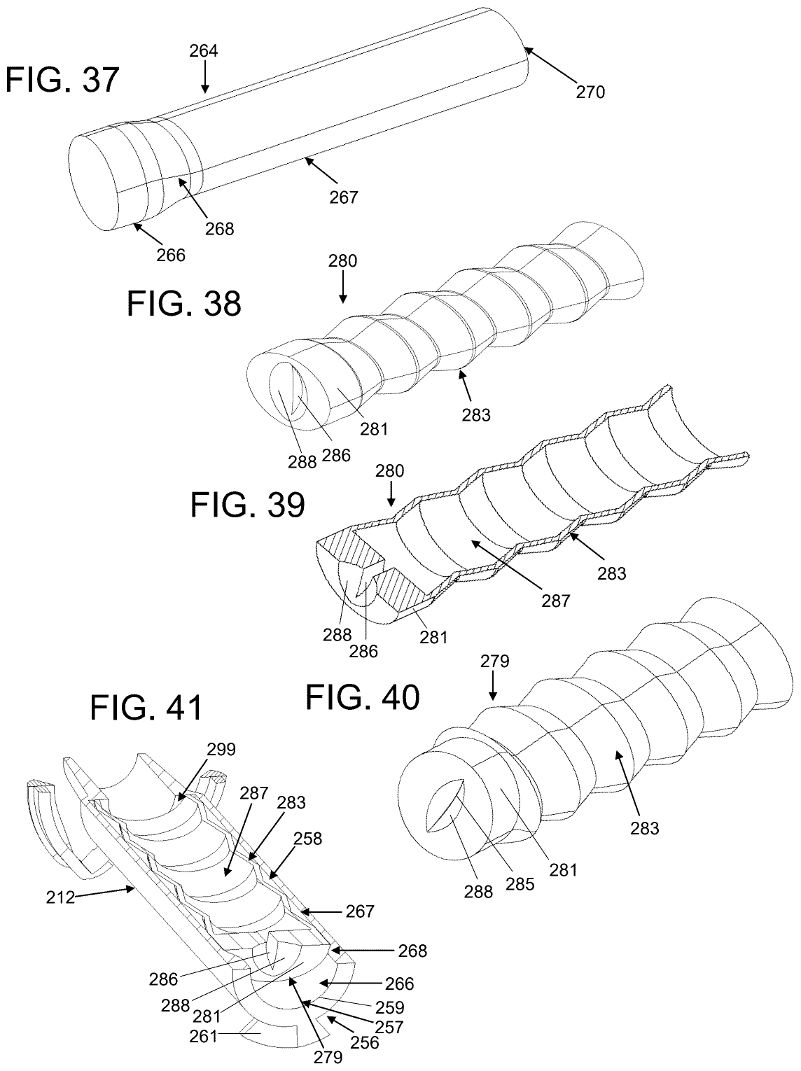

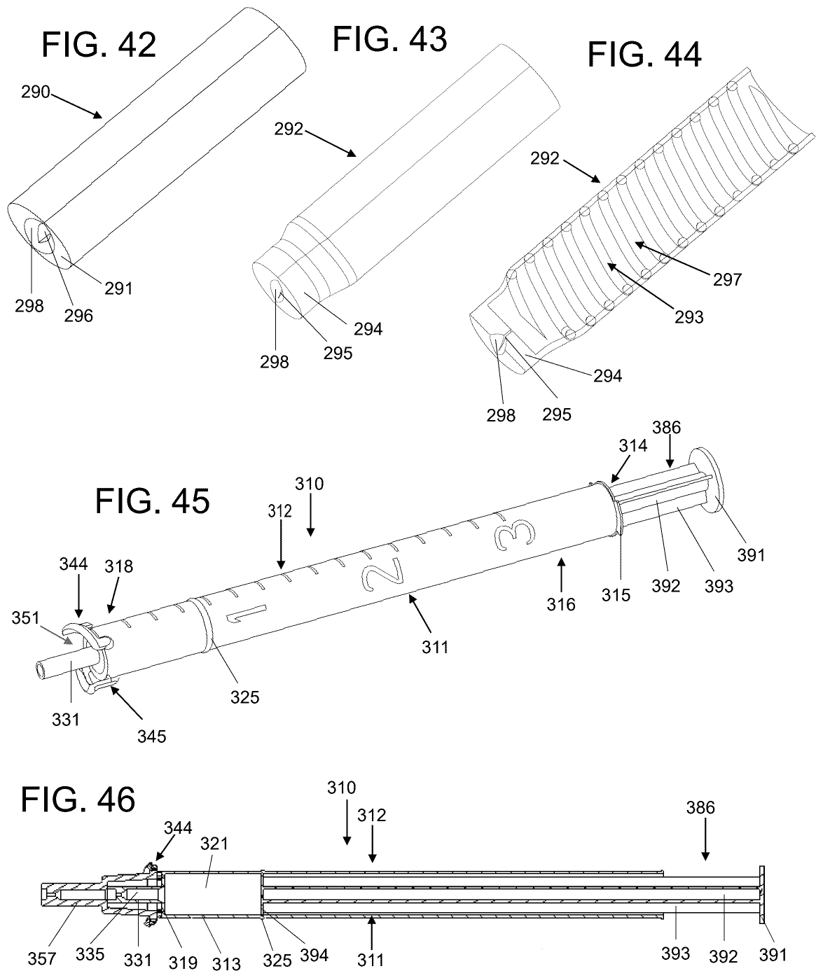

Referring now particularly to FIGS. 30, 31 and 32, another fitting/male Luer coupler 244 disclosure is for use with a fitting connector 210 for IV access ports providing one way flow and pressure protection, and instrument introduction. The distal end 218 supports a male, Luer coupler 244 consisting of two, opposing, rodded, cylindric sleeves 245, each with a partial locking thread 246 supporting inwardly sloping flank 247 encircling a central, hollow frustum 231's fluid passageway 235 with circumferential spacings 251. The lugs of an approaching female Luer coupler of another device may pass within the spacings 251 or be pressed passed the crests 255 of the sloped flanks 247 and locked with the underside flank 248 of the locking partial threads 244 with only a fractional turn for engagement without line entanglement. The external crest 254 of the locking thread 246 is an external thread and provides an attachment means for an accessory, such as a cap. The broad, conical taper 233 of the hollow frustum 231 allows approaching Luer hubs of varying pitched, internal tapers the ability to fluidly seal about the frustum 231 for contact is made about the inner rim of the hub's proximal opening. The connector fitting 210's proximal end 216 supports a female, Luer coupler 256 about the passageway 235 selectively occluded with an elastomeric, corrugated, elliptic check valve 280. The internal rim 259 of female Luer hub 257's cylindrical opening 260 provides a sealing edge for it is free of a mold parting line or flash due to its molding. Two opposing, helical Luer lugs 261 flank the hub 257 for coupling to a peripheral device. Referring now to FIGS. 33, 34, 35, 36, and 37, the fitting 210's internal geometry 258 in the valve chamber 265 distally transitions in region 268 from circular 266 to elliptical 267 in cross-section creating a subtle shoulder 268 at the major axis vertices 269 while maintaining a constant sectional area. This permits mold core retraction without permanent deformation while chamber is still thermally elastic. Referring to FIGS. 38 and 39, the elastomeric check valve 280 is an elliptical, corrugated, hollow cylinder distally closed with an integral, solid valve body 281 supporting a central, elliptical open passageway 286 with opposing axes. The compressive, transversely corrugated, distal sidewall 283 statically positions the valve body within the cylindric throat region 266 of fitting body 212 abutting the proximal end 250 of the male, Luer coupler 244 establishing the distal end of fluid chamber 270. The valve body 281 in FIG. 40, when compressed within the cylindric throat region 266, closes the central, elliptical slit passageway 285 and circumferentially seals within throat region 266. Now referring to FIG. 41, the insertion of a male, Luer member depresses valve body 289 into the elliptical body chamber 267 by axially compressing the corrugated sidewall 292 folding into the chamber void 215 and reforming the valve body 289 to elliptical, opening the elliptical slit passageway 290 permitting fluid flow into valve fluid channel 293 through the fitting 210. Upon removal of the male Luer member, the elastomeric valve 280 circularly reforms into the cylindric throat region 266, closing the passageway 285 and reestablishing its circumferential seal. The body 212's variance in cross-sectional geometry 268 and the valve 280's elastomeric material durometer retain valve 288's position and prevent expulsion from a slightly elevated blood pressure without a cross-sectional area stop. This configuration greatly eases assembly. The stepped, inwardly receding valve orifice 294 provides a visual guide and facilitates entry access, example, for a guide wire introduction assisting an endovascular procedure. Referring to FIG. 42, an alternative configuration for an elastomeric check valve is shown. The valve 290 is an elliptical, smooth, hollow, elastomeric cylinder with a proximal, integral, valve body 289 supporting a central, elliptical slit passageway 296 with opposing axes. The compressive, distal sidewall 295 supports an integrally molded, opposing, pair of helical threads reestablishes the valve 290's erectness. 43, and 44

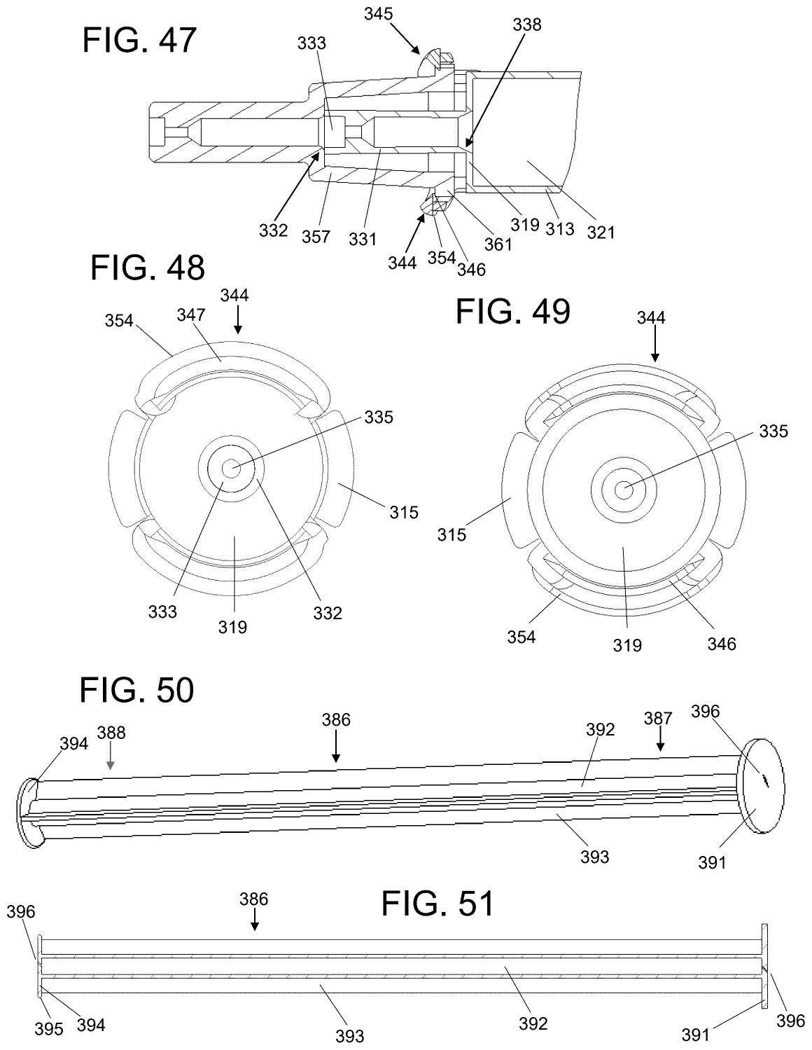

FIG. 45 reflects an axonometric view of another embodiment of a male Luer lock assembly 344 molded to the peripheral, distal end 318 of an injection molded body 311 permitting segmented, internal, locking and external thread details 346 and 354, respectively, on the rodded, Luer cylindric sleeves 345 to be molded with a single axis approach. Referring to FIGS. 46 and 47, the syringe body 312 is restricted to a diameter within the male Luer lock assembly 344, unless butterfly molded or as an insert. The concept supporting this disclosure is the adaptability offered for different functionalities. This particular syringe facilitates guide wire introductions for endovascular procedures. The coupling arrangement accepts a female Luer assembly or the cannula may be attached directly in the cannular socket. The male Luer coupling 344 uses a columnar conduit 331 with a distal sealing face 332 integrally molded into the end wall 319. The fluid seal is established either with the outer, distal edge of the conduit engaging the sloped inner wall or abutting the neighboring, stepped surface of the Luer hub. The sealing tension is established and maintained by the coupling of the helical locking thread 346 engaging the hub lugs 361. To couple, the Luer hub lugs 361 could be passed into the openings 351 between the circumferentially spaced, partial, locking threads 346 or pressed past the sloping flanks 347 of the locking threads within the yielding rodded sheaths 345 reaching the travel limit of the conduit 331 height, and then slightly twisted for locking securement. To decouple, twist lugs 361 to openings 351 and lift off; or, the Luer hub 357 may be laterally twisted to snap the fluid conduit 331 at its circumferential, thinned segment 338 free from the distal end wall 319 and outwardly forcing the slightly upward sloping thread flanks 348 to release the Luer lugs 361. The proximal barrel flange 315 is segmented for molding clearance with the male Luer coupler 344. FIGS. 48 and 49 are top and bottom views of syringe 312 revealing single axis molding is applicable. The plunger 386 is an extruded tube with a central tubular shaft 392 supporting three centering fins 393. Both ends 387 and 388 are “tipped” as described above, a proximal finger pad 391 and a distal fluid sealing disc 394 with sealing crown 395, and support arcuate, angled self-healing slits 396 providing access.

The various, above-described features can be interchangeable within the various embodiments, and by utilizing the various embodiments of the subject invention, a thermoformed syringe becomes produceable and sustainable. In particular, any element disclosed in one embodiment may be incorporated or utilized with any other embodiment disclosed herein.

Terms such as “attached,” “connected,” “coupled,” and variations thereof, as used herein, means the joining of two members directly or indirectly to one another. Such joining may be stationary (e.g., permanent or fixed) or moveable (e.g., removable or releasable). Such coupling may be mechanical or fluidic, for example.

The phraseology and terminology used herein is for the purpose of description and should not be regarded as limiting. The use of “including,” “comprising,” “having,” “containing,” “involving,” and variations thereof herein, is meant to encompass the items listed thereafter, equivalents thereof, and additional items, as well as alternate embodiments consisting of the items listed thereafter exclusively. In one embodiment, the devices described herein consist of one, each combination of more than one, or all of the described elements, acts, or components.

It should be noted that the orientation of various elements may differ according to other exemplary embodiments, and that such variations are intended to be encompassed by the present disclosure.

Further, references in the singular or plural form are not intended to limit the presently disclosed systems or methods, their components, acts, or elements to single or plural configurations.

Where technical features in the drawings, detailed description or any claim are followed by reference signs, the reference signs have been included to increase the intelligibility of the drawings, detailed description, and claims. Accordingly, neither the reference signs nor their absence has any limiting effect on the scope of any claim elements.

References to “or” may be construed as inclusive so that any terms described using “or” may indicate any of a single, more than one, and all of the described terms.

References to at least one conjunctive list of terms may be construed as an inclusive OR to indicate any of a single, more than one, and all of the described terms. For example, a reference to “at least one of ‘A’ and ‘B’” can include only ‘A’, only ‘B’, as well as both ‘A’ and ‘B’. Such references used in conjunction with “comprising” or other open terminology can include additional items.

Modifications of described elements and acts such as variations in values of parameters or variations in arrangements can occur without materially departing from the teachings and advantages of the subject matter disclosed herein. For example, elements shown as integrally formed can be constructed of multiple parts or elements, the position of elements can be reversed or otherwise varied, and the nature or number of discrete elements or positions can be altered or varied. Other substitutions, modifications, changes and omissions can also be made in the design and arrangement of the disclosed elements and operations without departing from the scope of the present disclosure

The devices described herein may be embodied in other specific forms without departing from the characteristics thereof. The foregoing embodiments are illustrative rather than limiting of the described devices. The scope of the devices described herein is thus indicated by the appended claims, and changes that come within the meaning and range of equivalency of the claims are embraced therein.

1. A syringe comprising:
a body tube having an open proximal end and a distal end closed with an insert comprising:
a chamber sealing disc supporting a fluid passageway enclosed by a male Luer coupler, the male Luer coupler comprising:
a first partial cylindrical sleeve, supporting an interior, partial, locking thread; and
a tube supporting a distal, fluid sealing disc and a finger pad at an opposite end moveably disposed within the tube.

2. The syringe as defined in claim 1, wherein the coupler further comprises; a second partial cylindrical sleeve opposing the first partial cylindrical sleeve with an opposing, partial, locking thread flight.

3. The syringe of claim 1, wherein the insert is butterfly molded and conjoined to provide
concealed thread flanks with single axis injection molding.

4. The syringe as defined in claim 1 and further comprising:
a flange on the open proximal end.

5. The syringe as defined in claim 1 and further comprising:
a hollow, frustum-shaped hub frictionally coupled with opposing, locking lugs extending peripherally about the base of the hub, the hub supporting an axially aligned cannula, wherein the hub and the locking lugs are in fluid alignment.

6. The syringe as defined in claim 5 and further comprising:
opposing, axial, thread voids within the first partial cylindrical sleeve created by partial thread circumferential
spacing, permitting passage of the hub lugs prior to engagement with a locking thread during coupling.

7. The syringe as defined in claim 5 and further comprising:
a pair of peripherally extending levers or flanges about the first and second partial cylindrical sleeves to initiate
decoupling by forcing the sleeves outward, thereby disengaging locking threads from the lugs.

8. The syringe as defined in claim 5 and further comprising:
an axially lower, circumferentially first thread offset from a locking second, ejector thread in the first partial cylindrical sleeve, assisting separation of cannula hub from syringe body, activated by outward movement of the first partial cylindrical sleeve.

9. The syringe as defined in claim 5 and further comprising:
a sheath assembly rotationally attached about the hub of the cannula via an integral tensioned yoke.

10. The sheath assembly as defined in claim 8 and further comprising:
a closed, tubular needle sheath with selective, cannular shielding postures established by a tensioned, levered yoke about the hub.

11. A syringe comprising:
an injection molded body with an open proximal end and a closed distal end supporting a fluid passageway; and
two opposing, peripheral cylindrical appendages about the distal rim each supporting an interior, partial locking thread; and
a tube supporting a distal, fluid sealing disc and a finger pad at an opposite end moveably disposed within the tube.

12. The syringe as defined in claim 11 and further comprising;
a second axially aligned, partial, ejection thread to allow decoupling of cannula hub from syringe body.

13. The syringe as defined in claim 11 and further comprising;
a flange on the open, proximal end of the body.

14. The syringe as defined in claim 11 and further comprising:
opposing openings between sleeves permitting passage of the hub lugs prior to engaging of a locking thread during coupling.

15. The syringe as defined in claim 11 with a fluid passageway comprising:
a hollow frustum with a circumferential, thinned wall segment, permitting fracturing with a lateral and/or twisting force.

16. A fitting comprising:
a tubular housing with a transitional interior chamber supporting a distal, external, peripheral male Luer coupler and a proximal, female Luer coupler with an elastomeric, directional valve element within.

17. The fitting as defined in claim 16, wherein the chamber is configured to transition from circular to elliptical to circular in cross-section, so as to create opposing, interference shoulders to retain the elastomeric, directional valve element.

18. The fitting as defined in claim 16, wherein the elastomeric valve element comprises:
a proximally occluded, elliptical, hollow cylinder supporting a central, compressively sealed orifice with
transversely corrugated sidewalls permitting axial flexure to allow the compressively sealed orifice to open upon engagement with the external, peripheral male Luer coupler.

19. The fitting as defined in claim 16, wherein the elastomeric fitting element further comprises
a proximally occluded, elliptical, hollow cylinder supporting a central compressed orifice with sidewalls integrally supporting opposing helical threads, permitting axial flexure to allow a seal of the compressed orifice to open upon engagement with the external, peripheral male Luer coupler.
